(19) United States
(12) Patent Application Publication
(10) Pub. No.: US 2015/0050730 A1
(43) Pub. Date: Feb. 19, 2015

(54) Process for Ex Vivo Expansion of Stem Cells in a Bioreactor

(71) Applicant: INSTITUTO SUPERIOR TÉCNICO, Lisboa (PT)

(72) Inventors: Joaquim Manuel Sampaio Cabral, Lisboa (PT); Claudia Alexandra Martins Lobato da Silva, Venda do Pinheiro (PT); Pedro Miguel Zacarias Andrade, S.Juliao do Tojal (PT); Francisco Ferreira dos Santos, Lisboa (PT); Maria da Graca Nortadas Duarte de Almeida-Porada, Winston-Salem, NC (US)

(21) Appl. No.: 14/387,318

(22) PCT Filed: Mar. 19, 2013
(86) PCT No.: PCT/PT2013/000017

§ 371 Date: Sep. 23, 2014

(30) Foreign Application Priority Data
Mar. 12, 2012 (PT) 106225

(51) Int. Cl. C12N 5/0775

(52) U.S. Cl. CPC C12N 5/0663; C12N 2502/1171; C12N 2500/92; C12N 2501/125; C12N 2501/26; C12N 2501/145; C12N 2501/115; C12N 2531/00
USPC 435/347; 435/289.1

(57) ABSTRACT

The present invention refers to a process of ex vivo expansion of stem cells, in a bioreactor, in particular hematopoietic stem/progenitor cells co-cultured with mesenchymal stem cells immobilized on microcarriers, for transplantation. The process comprises the steps of: a) forming a suspension of mesenchymal stem cells immobilized on microcarriers, b) inoculating in a bioreactor containing an expansion medium, hematopoietic cells co-cultured with mesenchymal stem cells immobilized on microcarriers c) expansion of hematopoietic cells. The process of the invention is capable of being implemented in a Kit.

### FIELD OF INVENTION

The present invention refers to a process for the ex vivo expansion of stem cells in a bioreactor, in particular, hematopoietic stem/progenitor cells co-cultured with mesenchymal stem cells immobilized on microcarriers, for transplantation.

### BACKGROUND OF INVENTION

Stem cells are cells capable of self-renewal and/or produce mature cells of different lineages in vitro and in vivo. Thus, the use of stem cells and their progenitors appears to be a promising strategy for clinical applications, particularly in cell and gene therapy envisaging the treatment of various degenerative diseases and/or as an adjuvant immunotherapy for the treatment of aggressive forms of cancer.

In particular, in bone marrow transplants, hematopoietic stem cells are the only stem cell therapy based on stem cells implemented worldwide.

Since the first bone marrow transplantation in 1968 the use of hematopoietic stem cells has increased exponentially, reaching 13,000 donations in 2008 with the aim of treating malignancies such as leukemia, lymphomas, myelomas, solid tumors (breast cancer, cancer testicular, etc.). The strategy for the treatment of malignant diseases involves the administration of high doses of chemotherapy and/or radiotherapy, while bone marrow transplantation promotes restoration of hematopoietic function (i.e., blood and immune system).

In the case of non-malignant diseases such as aplastic anemia, thalassemia, Gaucher's disease, etc, the dysfunctional patient's bone marrow is destroyed and replaced with a bone marrow of a healthy donor.

More recently, several clinical trials have begun for the use of hematopoietic stem cells for the treatment of non-hematopoietic disorders, such as, for example, hereditary epidermolysis bullosa, ischemic neonatal encephalopathy, acute myocardial infarction, amyotrophic lateral sclerosis and stroke, etc.

The most primitive hematopoietic cell expresses the surface antigen CD34, a transmembranar glycoprotein, associated to the adhesion of stem and progenitor cells in the bone marrow. The expression of CD34 antigen has been used as a criterion for the identification and isolation of hematopoietic stem/progenitor cells. The primitive cells are also identified based on the expression of the protein Thy-1 (or CD90) a marker related to T cells. Simultaneous expression of CD34 and CD90 on hematopoietic cells (ie, CD34+CD90+) resulted in efficient and sustained levels of engraftment. The co-expression of CD34 and CD90 is directly related to the potential of marrow repopulation.

Generally, the sources of hematopoietic stem cells include bone marrow, mobilized peripheral blood, cord blood and fetal liver. The cells of umbilical cord blood have unique characteristics compared with cells from bone marrow and peripheral blood, preferably, by being immature cells, with longer telomeres and, consequently, with a higher proliferative potential, with immediate availability after harvesting, without risk to the mother and/or baby. Moreover, these cells have a lower risk of contamination by viruses, allowing greater disparity of human leukocyte antigen (4/6 vs. 6/6 for bone marrow and peripheral blood), increasing the range of potential compatible donors.

A single unit of cord blood contains a limited number of cells for transplantation (about 5×10⁸ mononuclear cells), typically 100 times lower than the number obtained from the peripheral blood and 10 times smaller than those obtained from bone marrow. This is a limiting factor for therapeutic application, due to the fact that most patients transplanted with cells from cord blood are children with a weight between 20 30 Kg. Therefore, the functional performance of the hematopoietic graft will strongly depend on the cellular dose administered.

The ex vivo expansion appears then as an alternative in order to increase the number of cells of umbilical cord blood available for the hematopoietic transplantation. In this regard, the implementation of some clinical trials involving the use of hematopoietic stem cells from umbilical cord blood expanded ex vivo has shown promising results.

Indeed, interactions between stromal cells with hematopoietic stem cells are known and are considered crucial in maintaining the characteristics of multipotent hematopoietic stem cells after ex vivo culture. In fact, stromal cells, in particular human mesenchymal stem cells from bone marrow, have been used in co-culture with hematopoietic stem cells from the umbilical cord blood, resulting in improved expansion of hematopoietic stem cells, as well as a superior preservation of the quality of the graft during ex vivo culture.

However, the use of stroma to support the expansion of hematopoietic stem cells confers some limitations to the process, especially considering the complexity of the culture system that necessarily must accommodate the elements of the stroma (surface adherent cells) and hematopoietic stem cells (suspension cells). This limitation is greater the larger the scale of production required of hematopoietic stem cells.

Because the hematopoietic stem cells are located in vivo in the bone marrow, a tissue which is essentially static, with very low flows, which allows a stable and lasting contact between the various cell types, molecules and matrices, the culture conditions considered ideal would be those able to closely mimic the hydrodynamic conditions of the environment of the bone marrow.

The cultures of stem cells, in particular hematopoietic stem cells have been effected under static conditions, typically in traditional culture flasks (eg petri dishes or tissue culture flasks) that are limited in terms of cellular productivity, in their non-homogeneous nature, without monitoring culture parameters and with highly required manipulation upon feeding and/or recovery procedures of cells in culture.

The international application WO 2010/138873 refers to a process for expansion of hematopoietic stem cells comprising co-culture with mesenchymal stem cells in the presence of growth factors. The conditions for expansion of hematopoietic stem cells are based on static systems, in which the cellular expansion is provided in layers.

Therefore, alternative systems are highly desirable that provide higher cell productivities, monitoring and control parameters assigned to the culture, such as pH, temperature, dissolved oxygen concentration, and others with the possibility of scaling up.

Several types of bioreactors systems for ex vivo expansion of hematopoietic stem cells have been tested: mechanically agitated systems (7-fold increase in the number of stem cells after 28 days of initial culture), rolling bottle systems (17-fold increase in the number of colony forming units—CFU), perfusion chambers (2.4 fold increase in the number of total cells, no increase in CD34+ progenitor cells after two weeks in culture). While not exhaustive, this set of results represents a limited potential for therapeutic application not only for the supplementation of culture media with animal products (eg. fetal calf serum) as well as due to the low number of cells obtained.

Kedong et al. have obtained an expansion factor of 5 in CD34+ cells population starting from a non-enriched (mononuclear fraction) after 12 days in culture in a roller bottle device. Moreover, Jaroscak et al. have obtained an expansion factor of 2.4 in total hematopoietic cells, in the same time period, using an automated perfusion process, although no significant expansion of CD34+ cells was observed.

The international application WO 2008/149129 discloses a process using ex vivo expansion of CD34+ progenitor cells from umbilical cord blood, in which these cells are encapsulated in a supportive matrix and placed in a bioreactor to provide its expansion. However, it is apparent that in the process disclosed herein, the cellular expansion is confined to the beads so that constitute the backing layer, thereby limiting cell growth.

In accordance with the foregoing, a need exists to develop a process of ex vivo expansion of stem cells in dynamic culture systems, scalable and highly monitored and controlled, so as to achieve a high cell productivity with unrestricted cell growth, with compliance to the Good Manufacturing Practice (GMP) that can adjust to the quality parameters required by regulators.

### SUMMARY OF THE INVENTION

The process of ex vivo expansion of stem/progenitor of the present invention comprises:

a) forming a suspension of mesenchymal stem cells immobilized on microcarriers,

and further comprising:

b) inoculation in a bioreactor containing an expansion medium, hematopoietic cells co-cultured with mesenchymal stem cells immobilized on microcarriers, and

c) expansion of hematopoietic cells.

In one aspect, the process is characterized in that the mesenchymal stem cells are bone marrow cells.

In another aspect of the invention, the process is characterized in that the cells are hematopoietic cells from umbilical cord blood.

In yet another aspect, the process of the invention is characterized in that said hematopoietic cells from cord blood are enriched for CD34 antigen prior to step b) of inoculation.

Preferably, the ratio of mesenchymal stem cells and hematopoietic stem cells is 2:1.

Also preferably, step c) of expansion takes place in a time interval of expansion of 4 to 14 days, more, preferably between 7 and 14 days and most preferably within 10 days.

The process of the invention is characterized in that the step c) of expansion comprises:
alternate cycles of 1 to 10 minutes of stirring between 10 rpm and 100 rpm, followed by 2-6 hours of rest during the first day and
constant stirring speeds from 10 rpm to 100 rpm in the following days.

In a most preferred embodiment of the present invention, the process comprises cycles of 5 minutes stirring at 40 rpm, followed by 4 hours of rest, during the first day and constant stirring at 40 rpm, the following days.

Preferably, the expansion medium of hematopoietic cells is a serum-free medium and comprising cytokines.

In a preferred aspect of the invention, said cytokines are selected from the group comprising stem cell factor (SCF) fms-related tyrosine kinase 3 (Flt-3), thrombopoietin (TPO) and fibroblast growth factor (FGF) and combinations thereof.

Most preferably, the dissolved oxygen concentration in the expansion medium is between 0.30 mg/L and 7.50 mg/L, the pH of the expansion medium is between 7.0 and 7.5 and the temperature of the expansion medium is between 36° C. and 38° C.

### BRIEF DESCRIPTION OF THE DRAWINGS

As follows, it is presented the detailed description of the invention with reference to the accompanying drawings, in which:

FIG. 1 shows graphically the expansion, under dynamic conditions, in a flask equipped with means for stirring of total hematopoietic cells and CD34+ cells from cord blood co-cultured with mesenchymal stem cells, immobilized on microcarriers (condition with mesenchymal stem cells—“with MSC”). As a control, hematopoietic cells from umbilical cord blood were cultured in flask equipped with means for stirring, in the absence of mesenchymal stem cells immobilized on microcarriers (condition without mesenchymal stem cells—“No MSC”).

FIG. 2 graphically depicts the expansion, under dynamic conditions in a flask equipped with means for stirring of CD34+ cells from umbilical cord blood co-cultured with mesenchymal stem cells, immobilized on microcarriers (condition “Flask equipped with means for stirring) and static conditions (“static+MSC”). It was also performed a static control without mesenchymal stem cells immobilized on microcarriers (condition “Static no MSC”). The levels of expansion or proliferation represented by the expansion factor were determined during the culture time for all three conditions.

### DETAILED DESCRIPTION OF THE INVENTION

The present invention relates to a process of ex vivo expansion of stem cells in bioreactor, hematopoietic stem/progenitor cells co-cultured with mesenchymal stem cells immobilized on microcarriers, for transplantation.

Surprisingly it has been found that the process of the present invention provides rates of expansion of hematopoietic cells superior to prior art in dynamic conditions.

This result is unexpected since the skilled in the art might reasonably expect that a bioreactor type of culture presents drawbacks resulting from shear stress induced by agitation, which shear stress is considered detrimental to the maintenance/viability of animal cells, in particular hematopoietic stem cells. Moreover, the poor results obtained by dynamic processes (in bioreactor) mentioned above were attributed to these reasons by the skilled in the art.

Unless otherwise stated, the ranges of values given herein are intended to provide a simplified and technically acceptable manner to indicate each individual value within the respective range. For example, the phrase “1 to 2” or “between 1 and 2” means any amount within this range, for example 1.0, 1.1, 1.2, 1.3, 1.4, 1, 5, 1.6, 1.7, 1.8, 1.9, 2.0. All figures quoted herein should be interpreted as approximate values, for example the reference to “2.0” means “approximately 2.0”.

Note that regardless of explicit presentation of a quantitative expression “about X” means any value X submitted during the present description must be interpreted as an approximate value of the actual value X, since such an approximation to the actual value would be reasonably expected by a skilled in the art due to experimental conditions and/or measurements which introduce deviations to the actual value.

As used herein, the terms “mesenchymal stem cells”, “stromal cells” and “supportive cells” describe multipotent stem cells, originating from various human tissues that are covered by the criteria established in 2006 by the International Society of Cell Therapy:

1) cells in adherent cell culture flasks are in traditional culture conditions;

2) more than 95% of the cell population must express the markers CD73, CD90 and CD105, as measured by flow cytometry and does not express, or express less than 2%, the markers CD11b or CD14, CD34, CD45, CD79a or CD19 and HLA Class II;

3) cells are differentiated into osteoblasts, chondroblasts and fat cells in suitable in vitro culture conditions.

The process of cell expansion of the present invention begins with the formation of a supportive layer from isolation of mesenchymal stem cells. These cells can be isolated from various adult tissues such as bone marrow, umbilical cord blood, umbilical cord array, adipose tissue, amniotic fluid or urine.

In one embodiment of the present invention, mesenchymal stem cells isolated from bone marrow are used. These cells are expanded under static conditions and then added to a bioreactor with the microcarriers, previously prepared. As a result, they form a cell suspension immobilized on microcarriers to which is added subsequently, a compound that inactivates the growth of mesenchymal stem cells.

By “hematopoietic stem cells”, “stem cells” and “primitive cells” refers to hematopoietic stem cells capable of proliferation and in vivo repopulating the bone marrow of a immunocompromised mammal (i.e., an individual who has an impaired immune system or absent when transplanted).

It is known that the most primitive hematopoietic cell expresses the surface antigen CD34, which is a transmembranar glycoprotein, linked to the adhesion of stem/progenitor cells to the bone marrow. The expression of CD34 antigen has been used as a criterion for the isolation and identification of hematopoietic stem/progenitor cells. The primitive cells are also identified based on the expression of the protein Thy-1 (or CD90) a marker related to T cell. Simultaneous expression of CD34 and CD90 on hematopoietic cells (i.e., CD34+CD90+ cells), resulted in efficient and sustained levels of engraftment. The co-expression of CD34 and CD90 is directly related to the potential of marrow repopulation of the transplanted mammal.

The terms “total hematopoietic cells” and “total cells” refer to all hematopoietic cells (stem and non-stem, i.e. primitive and mature) in culture. In a particular manner, mature cells (also known as differentiated cells) may comprise CD3+, CD19+, CD14+, CD15+, CD33+, CD11c+HLA-DR+, CD56+, CD235a+, CD41+, CD38+, CD45RA+ and CD127.

In the present invention, the culture of hematopoietic stem cells and mesenchymal stem cells is termed co-culture, as regards to the culture of two types of different cells in the same bioreactor.

In the present description, the term “bioreactor” means a reaction vessel equipped with stirring and control means suitable for biological reactions to occur under dynamic conditions. It is considered dynamic conditions all of those which are not solely static including, for example, alternating agitation cycles with rest states.

The relative amount of hematopoietic stem cells and mesenchymal stem cells in the beginning of the co-culture is designated by reason of mesenchymal stem cells and hematopoietic stem cells. The two types of stem cells in co-culture may belong to the same donor or different donors.

The terms “cell expansion” or “cell proliferation” refer to the increase in cell population (e.g., hematopoietic stem cells) from the initial number of cells in culture as a result of maintaining suitable culture conditions to promote cell division. In order to quantify this expansion, it was used an “expansion factor” which is the result of the quotient of the number of cells in a certain day and the number of cells at the beginning of cell culture. For example, an expansion factor of 10 in CD34+ cells means that the original population increased ten times the original number of cells.

In one embodiment of the invention, the hematopoietic stem cells are cells harvested from umbilical cord blood, enriched for CD34 antigen before being placed in co-culture, in a bioreactor, with mesenchymal stem cells of the supportive layer.

The skilled in the art can select any bioreactor within the scope defined in the present invention. Bioreactors with different configurations may be considered in the context of animal cell culture, for example and not limited to, stirred tank reactor, fixed bed reactor, fluidized bed reactor and wave reactor. The stirred tank type reactors operate by mechanical action of a turbine or blade, conferring a homogeneous environment to the cell culture. On the other hand, fixed bed and fluidized bed reactors comprise the presence of a pack (or bed) usually composed by inert materials (e.g., synthetic carriers, polymers) of high area per unit volume and that promote cell adhesion (e.g., polystyrene, polycaprolactone, poly (lactic-co-glycolic acid) etc.), allowing lower operating flow rates and, consequently, lower shear forces, thus reducing the deleterious impact on cell viability. Similarly, the wave bioreactor promotes similar dynamic conditions through the oscillation of a biocompatible bag, commercially available from GE Healthcare, containing the various components of co-culture. Preferably, the reactor used in the present invention is a stirred tank type reactor, equipped with means for stirring.

The present invention further comprises a step of inoculation. At the beginning of this step the cell density of hematopoietic stem cells is 5×10⁴ cells/ml. These cells are thus loaded in a bioreactor together with the cells of the supportive layer.

In one embodiment the ratio of mesenchymal stem cells and hematopoietic stem cells is 2:1.

As will be understood by the skilled in the art, to ensure optimal culture conditions for ex vivo expansion of stem cells, the process of the present invention further comprises physical and chemical stimuli.

The physical stimuli to monitor and control are: concentration of dissolved oxygen, which should range between 0.30 mg/L and 7.50 mg/L, preferably between 0.33 mg/L and 7.1 mg/L, the pH of the culture medium, which should be between 6.5 and 8, preferably between 7 and 7.5, most preferably between 7.2 and 7.4, and the temperature, should be kept between 33° C. and 38° C.; preferably between 36° C. and 38° C., more preferably between 36.5° C. and 37.5° C., most preferably to 37° C.

The chemical stimuli include the addition of growth factors or cytokines, which are a group of proteins which occur naturally in vivo and which are necessary for the maintenance of ex vivo cultures. Growth factors, such as, stem cell factor (SCF), fms-related tyrosine kinase 3 (Flt-3), thrombopoietin (TPO), fibroblast growth factor (FGF), interleukin 1, interleukin 2, interleukin 10, interleukin 6, angiopoietin, leukemia inhibitory factor are known, solely or in combination, as promoters of ex vivo proliferation of hematopoietic stem cells.

Preferably, the cytokines to be used in the present invention must be selected from the group consisting of stem cell factor (SCF) receptor tyrosine kinases fms-like 3 (Flt-3), thrombopoietin (TPO) and fibroblast growth factor (FGF).

The cell expansion medium to be used is, preferably, a serum-free medium (which ensures the absence of immunological reactions due to the absence of animal proteins) supplemented with cytokines, which provides expansion of hematopoietic stem cells co-cultured with mesenchymal stem cells isolated on microcarriers.

The present invention also comprises an expansion step, wherein said bioreactor is subjected to a regime of stirring, in alternate cycles comprising in the first day of expansion, 1 to 10 minutes of stirring and 2 to 6 hours of rest, preferably 2 to 8 minutes of stirring and 3 to 5 hours of rest. In a most preferred embodiment, the regimen comprises, on the first day of expansion, 5 minutes stirring at 40 rpm and 4 hours of rest. In the remaining days, the stirring speed should be kept constant in a range of 10 to 100 rpm, preferably 20 to 80 rpm, more preferably 30 to 70 rpm. In the most preferred embodiment, the stirring speed is 40 rpm.

Regarding the expansion time, this will vary between 0.4 and days, preferably between 7 and 14 days, most preferably 10 days.

During the cell expansion the expansion medium is replaced, for example, on days 3, 7 and 10, to allow the continued expansion of cells, without saturation of the medium.

According to the process of the present invention, in the end of the cell expansion step, the interior of the bioreactor contains liquid medium with total hematopoietic cells, hematopoietic stem/progenitor cells as well as inert carriers containing mesenchymal stem cells adhered to the surface.

Then, based on the difference in diameter between hematopoietic cells (2-10 mm) and the inert carriers (greater than 100 microns) it is possible to separate these two components using a filter with a pore diameter greater than 10 mm and less than 100 micrometers.

In a surprising way, in a preferred embodiment, clinically significant figures were obtained of 19 million CD34+ cells, as a result of the cell expansion process of the invention. These results overcome the limitations observed in the prior art, since they allow one to obtain sufficient number of cells for transplantation in adults, for example, to reconstitute, in a stable and lasting manner, the blood system when transplanted into humans.

The present invention can be implemented in the form of a kit comprising a cell culture bag, with variable volume (e.g. at least two compartments separated by seals) containing in a first compartment, mesenchymal stem cells (MSC) immobilized on microcarriers and, after selection/enrichment (done at this time) of the cells of umbilical cord blood for the CD34 surface antigen, they are inoculated in a bioreactor with a culture medium supplemented with cytokines (2nd component of the Kit). At this time, intercommunication between the two compartments is allowed as well as the establishment of the co-culture.

This disposable and single use cell culture bag, is placed in a bioreactor, for example, the wave bioreactor (stirring speeds between 20-60 rpm—preferably 40 rpm) during the culture time (4-14 days, preferably 10 days), in which, at the end of the culture, the cell suspension is passed through an outlet valve to which is coupled a filter with pore diameter greater than about 10 micrometers (typical sizes of hematopoietic stem cells HSC in culture) and less than about 100 microns (minimum diameter of microcarriers+MSC), so that the cells of interest may be collected and administered.

This kit has the advantage of having the possibility of being stored/shipped frozen (e.g., at −196° C. in liquid nitrogen or dry ice, respectively), the bag (containing the cells/microcarriers) that can be thawed in a thermostatized water bath at a convenient temperature, for example 37° C., at the desired time to begin the co-culture, saving about 15 days total time for obtaining the dose of HSC, resulting from the elimination of the time for the establishment of the supportive layer of MSC.

For a better understanding of the invention is described below, by way of illustration and not limitation, an example of applying the process of the present invention.

### Example

### Establishment of the Stromal Layers Consisting of Mesenchymal Stem Cells Isolated from Bone Marrow Using Microcarriers in Flasks Equipped with Means for Stirring

Plastic, non-porous microcarriers, (SoloHill Engineering, Inc.) were prepared according to the manufacturer's instructions, coated with CELLstart™ (diluted 1:100 in PBS with Ca2+ and Mg2+) for 2 hours at 37° C. with intermittent stirring (1 minute at 300 rpm, 10 minutes off) using a thermostatized stirring block.

The human mesenchymal stem cells, previously isolated from a bone marrow aspirate (after centrifugation with Ficoll), were expanded for two passages in static conditions (standard culture flasks) using serum-free medium and added to 20 g/L of microcarriers, previously prepared. Then, were transferred to a flask equipped with means for stirring (Bellco Glass, Inc.) with a working volume of 80 mL, equipped with 90° blades and a magnetic stirrer. After day 3, it was retrieved, every day, 25% of the volume of medium and replaced by the same amount of fresh medium until day 10 of culture. The number of mesenchymal stem cells was determined in a 1 mL sample of the flask equipped with means for stirring, using the Trypan Blue method, after enzymatic digestion with Accutase (Sigma, 7 minutes at 37° C.) to release the cells from the plastic microcarriers. It was added Mitomycin C (Sigma), 0.5 ng/ml in Iscove's Modified Dulbecco's medium Medium (IMDM) to the suspension of cells immobilized on microcarriers, in order to inactivate the growth of mesenchymal stem cells.

Ex Vivo Expansion of CD34+ Enriched Cells in a Flask with Means for Stirring

The mononuclear fraction of umbilical cord blood was obtained after centrifugation with Ficoll gradient. Then, cells were enriched for the CD34 antigen using immunomagnetic particles with immobilized anti-CD34 (MACS, Miltenyi) (initial percentage of CD34+ cells, 84±3%).

The population of cells enriched for CD34 was inoculated into flasks equipped with means for stirring of 25 ml (Wheaton Science; working volume=20 mL) at a cell density of 5×10⁴ cells/ml in co-culture with mesenchymal stem cells immobilized on microcarriers (ratio of mesenchymal stem cells:hematopoietic stem cells 2:1).

During the first 24 hours, an intermittent stirring scheme was used: 5 minutes stirring at 40 rpm and 4 hours of rest during the first day of expansion, after which the stirring was set at 40 rpm until day 14.

During the expansion time, several physical parameters are monitored and controlled: oxygen tension (or percentage of oxygen), which should be between 0.33 mg/L and 7.1 mg/L of dissolved oxygen and which can be adjusted by injecting nitrogen, the pH of the culture medium, which should be between 7.2 and 7.4 and which can be adjusted by adding a solution of a base, such as NaOH, and temperature should be kept between 36.5° C. and 37.5° C., by circulating a fluid in the bioreactor jacket.

A serum-free medium was used, QBSF-60 (Quality Biological Inc.) supplemented with a combination of cytokines, with no components of animal origin, which was optimized for expansion of CD34+ cells from umbilical cord blood in co-culture with mesenchymal stem cells. Cytokines used were SCF at 60 ng/mL Flt-3 at 55 ng/mL TPO at 50 ng/ml bFGF and 5 ng/ml from Peprotech. The co-culture was fed on days 3, 7 and 10 of the expansion, removing half of the culture medium of the flask equipped with means for stirring and replacing the same quantity of fresh culture medium, ensuring that the volume remains constant throughout time in culture (14 days).

It was further carried out a control condition with the absence of mesenchymal stem cells immobilized on microcarriers (“No MSC”) retrieving a 1 mL sample of the flask equipped with means for stirring, filtering it (to separate hematopoietic cells from microcarriers with immobilized MSC) and then proceeded to count the number of total cells. Flow cytometry studies have been conducted to assess the number of hematopoietic stem/progenitor cell numbers (CD34+).

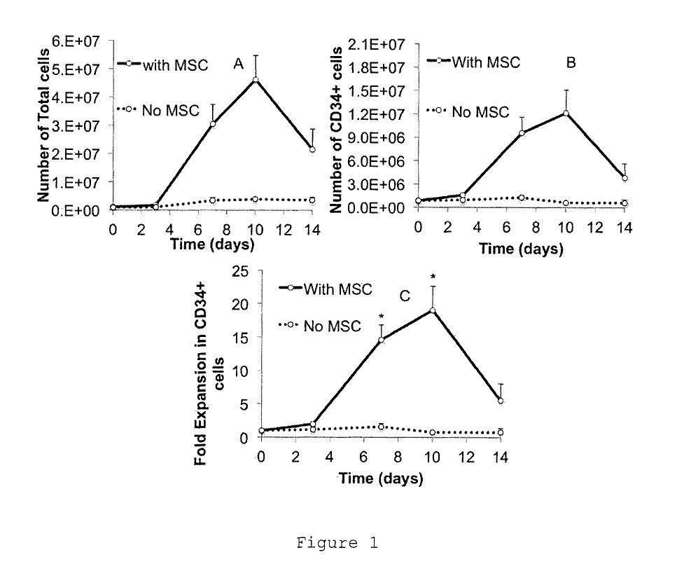

FIG. 1 shows levels of expansion of total hematopoietic cells and CD34+ cells in a flask equipped with means for stirring in the presence of mesenchymal stem cells immobilized on microcarriers (condition “With MSC”), yielding a total of 4.6×107 total cells and 1.49×107 CD34+ cells, corresponding to an expansion factor of 19.

The expansion factor was also determined over time in culture using a control without added MSC immobilized on microcarriers (condition “Without MSC”), where the expansion of total cells and CD34+ was not significant (expansion factor more than 1, 7 after 7 days in culture).

In order to evaluate the functional capacity of the cells obtained, it was monitored the potential for the formation of “cobblestone area forming cell” (CAFC) colonies of fresh cells in vitro (i.e. uncultivated) and expanded at day 7 and 10 culture, under stirred conditions (condition “flask equipped with means for stirring”) and static (“static”), in the presence of mesenchymal stem cells immobilized on microcarriers, as shown in Table 1 below.

TABLE 1
Potential for the formation of “cobblestone area forming
cell” (CAFC) colonies. The results are presented as
expansion factor (relative to day 0 - fresh cells) of CAFC
regarding the stirred conditions (flask equipped with means for
stirring, n = 4) and static (static, n = 1), both in the presence
of mesenchymal stem cells immobilized on microcarriers.

| | | | |
|---|---|---|---|
| | | flask equipped with | |
| CAFC | Day | means for stirring | Static |

| | | |
|---|---|---|
| 0 | 1.0 | 1.0 |
| 7 | 9.5 ± 0.70 | 5.5 |
| 10 | 4.9 ± 0.28 | 0.71 |

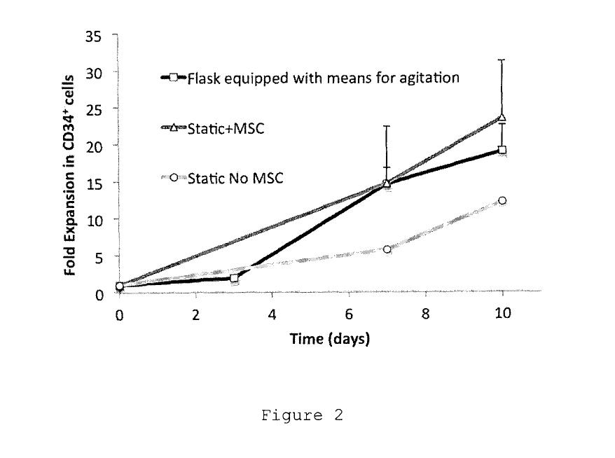

It was found that the culture flask equipped with means for stirring (under mechanical stirring) promoted a result of expansion similar to static condition (expansion factor of CD34+ cells of 19 versus 23) and considerably higher than the expansion in absence of mesenchymal stem cells under static conditions (expansion factor of CD34+ cells of 12 at day 10), as seen in FIG. 2.

However, although the values of CD34+ cell expansion obtained in dynamic conditions are similar to those of static systems, for an initial density of 5×10⁴ cells/mL it was obtained a clinically significant number of CD34+ cells (19 million) for a hematopoietic transplant in an adult patient.

These results constitute one of the major expansion performances of hematopoietic cells cultured in dynamic bioreactor (both in expansion factor and in number of cells produced) found in the literature, after 10 days of expansion (1.5×10⁷ CD34⁺ cells obtained from 1×10⁶ cells enriched for CD34+).

From the functional point of view, results are also positive, since the potential of forming CAFC obtained under dynamic conditions provided an expansion factor significantly greater than that obtained under static conditions at days 7 and 10 of culture.

1. Process for rapid expansion of number of hematopoietic stem and progenitor cells, characterized in that it comprises simultaneously:
a) providing a bioreactor system under stirred dynamic conditions;
b) providing a co-culture of hematopoietic stem cells with mesenchymal stem cells on inert supports, in the presence of growth factors in serum free medium, constituting a cell culture;
c) maintaining the cell culture in said bioreactor under dynamic conditions such that the expansion of hematopoietic stem cells is at least 5-fold in less than 20 days.

2. Process according to claim 1 characterized in that it comprises mesenchymal stem cells isolated from bone marrow or umbilical cord blood, or from umbilical cord matrix or adipose tissue or amniotic fluid, or urine.

3. Process according to claim 1 characterized in that it comprises mesenchymal stem cells frozen or fresh immobilized on inert supports.

4. Process according to claim 1 characterized in that it comprises hematopoietic stem/progenitor cells isolated from umbilical cord blood, bone marrow, mobilized peripheral blood and fetal liver.

5. Process according to claim 1 characterized in that it comprises mesenchymal stem cells of human origin.

6. Process according to claim 1 characterized in that it comprises mesenchymal stem cells and hematopoietic stem cells from the same donor.

7. Process according to claim 1, characterized in that it comprises the use of culture medium with no components of animal origin.

8. Process according to claim 1, characterized in that it adds the following growth factors: Stem Cell Factor (SCF), fms-related tyrosine kinase 3 (Flt-3), thrombopoietin (TPO) and fibroblast growth factor (FGF), interleukin 1, interleukin 2, interleukin 10, interleukin 6, Angiopoietin, leukemia inhibitory factor, solely or in combination.

9. Process according to claim 1, characterized in that it uses a stirred tank reactor, a fixed bed reactor or a fluidized bed reactor or a wave reactor, or other dynamic culture system for the cultivation of animal cells.

10. Process according to claim 1 characterized in that it has a ratio, at the beginning of culture, of mesenchymal stem cell to hematopoietic stem/progenitor cell of 2:1.

11. Process according to claim 1, characterized in that the cells obtained after expansion express the surface antigen CD34, CD90, or a combination thereof.

12. Process according to claim 1, characterized in that it comprises alternating cycles of 1 to 10 minutes stirring followed by 2 to 6 hours of rest during the first day of co-culture.

13. Process according to claim 12 characterized in that it has a constant stirring speed between 20 to 80 rpm after the first 24 hours of culture.

14. Process according to claim 1 characterized in that it monitors the dissolved oxygen concentration in the bioreactor between 0.33 mg·L⁻¹ and 7.1 mg·L⁻¹.

15. Process according to claim 1 characterized in that it has a pH control at 7.2 and temperature at 37° C.

16. Process according to claim 1 characterized in that after the period of co-culture, the reactor content is filtered, being the filtrate enriched for hematopoietic stem/progenitor cells.

17. Process according to claim 1 characterized in that the expanded population of hematopoietic stem/progenitor cells is positive for the reconstitution of the blood system test, when transplanted into a mammal.

18. Process according to claim 17 characterized in that the expanded hematopoietic stem/progenitor cells yield mature cells with the expression of surface antigens CD3+, CD19+, CD14+, CD15+, CD33+, CD11c+, HLA-DR+, CD56+, CD235a+, CD41+, CD38+, CD45RA+, and CD127+.

19. Process according to claim 18, characterized in that the mammal receiving the expanded hematopoietic stem/progenitor cells develop cells with the expression of surface antigens CD3+, CD19+, CD14+, CD15+, CD33+, CD11c+, HLA-DR+, CD56+, CD235a+, CD41+, CD38+, CD45RA+ and CD127+.

20. Culture of hematopoietic stem/progenitor cells characterized in that it is produced by the process described in claim 1.

21. Kit for cell growth by the process defined in claim 1, characterized in that it comprises a cell culture bag having at least two compartments separated by sealants with intercommunication between the said compartments, the first compartment containing mesenchymal stem cells immobilized on microcarriers, the second compartment medium supplemented with the cytokines and umbilical cord blood cells enriched for CD34 surface antigen.

22. Kit according to claim 21 characterized in that the culture medium used is supplemented with the following growth factors: Stem Cell Factor (SCF), fms-related tyrosine kinase 3(Flt-3), thrombopoietin (TPO) and fibroblast growth factor (FGF), interleukin 1, interleukin 2, interleukin 10, interleukin 6, Angiopoietin, leukemia inhibitory factor, solely or in combination.

23. Kit according to claim 21 characterized in that the microcarrier-immobilized cells are mesenchymal stem cells isolated from bone marrow or umbilical cord blood or from umbilical cord matrix, or adipose tissue, or the amniotic fluid, or urine.
